(19) United States
(12) Patent Application Publication
(10) Pub. No.: US 2013/0101575 A1
(43) Pub. Date: Apr. 25, 2013

(54) LACTOFERRIN SEQENCES, COMPOSITIONS AND METHODS FOR CORNEAL WOUND TREATMENT

(71) Applicants: Benjamin David Ashby, Collaroy Plateau (AU); Qian Garrett, Barden Ridge (AU); Mark Willcox, Balmain (AU)

(72) Inventors: Benjamin David Ashby, Collaroy Plateau (AU); Qian Garrett, Barden Ridge (AU); Mark Willcox, Balmain (AU)

(73) Assignee: Brien Holden Vision Institute, Sydney, New South Wales (AU)

(21) Appl. No.: 13/806,247

(22) PCT Filed: Jul. 1, 2011
(86) PCT No.: PCT/AU11/00826

§ 371 Date: Dec. 21, 2012

(30) Foreign Application Priority Data
Jul. 1, 2010 (AU) 2010902932

(51) Int. Cl. A61K 38/48

(52) U.S. Cl. CPC A61K 38/482
USPC 424/94.64; 435/219

(57) ABSTRACT

The present invention relates to pharmaceutical compositions containing lactoferrin, or fragments of it, and their use in the treatment of wounds, particularly corneal wounds. The present invention also provides a pharmaceutical composition comprising an effective amount of a polypeptide or peptidomimetic consisting essentially of the C-lobe of lactoferrin, or functionally active fragments or variants thereof.

### FIELD OF THE INVENTION

The present invention relates to pharmaceutical compositions containing lactoferrin, or fragments of it, and their use in the treatment of wounds, particularly corneal wounds.

### BACKGROUND OF THE INVENTION

The cornea is the transparent front part of the eye that covers the pupil, iris and anterior chamber. One of the important functions of the cornea is to maintain normal vision by refracting light onto the lens and retina. The human cornea is composed of five layers, of which the corneal epithelium is the anterior-most layer and forms the surface of the cornea.

The epithelial layer is predominantly cellular, composed of cells called keratinocytes. This layer acts as a physical barrier preventing, for example, microbial invasion of the deeper, more vulnerable structures. The stroma is underneath the epithelium and is made predominantly of collagen. It also contains other cells called keratocytes, which may play a role in stromal wound healing.

The ability of the cornea to maintain normal vision by refracting light onto the lens and retina is dependent in part on the ability of the corneal epithelium to undergo continuous renewal. Epithelial renewal is essential because it enables this tissue to act as a barrier that protects the corneal interior from becoming infected by noxious environmental agents. The renewal process also maintains the smooth optical surface of the cornea. This rate of renewal is closely maintained by an integrated balance between the processes of corneal epithelial proliferation, differentiation and cell death.

Damage to the corneal epithelium can be caused by foreign bodies (e.g. sand and grit), microbial insult or chemical insult, during contact lens wear or by surgery. Most corneal epithelial wounds heal promptly. However, in some cases, such as chemical injury, healing of the corneal epithelium is delayed, leaving the underlying stroma vulnerable to infection and ulceration. In addition, the eye is not able to maintain normal hydration, leading to cloudiness that reduces vision.

Alkali injuries are of particular concern and cause acute inflammation characterized by rapid infiltration of neutrophils into the cornea, followed by chronic inflammation, which involves the migration and recruitment of inflammatory cells over extended periods, further damaging the corneal surface. In serious cases this leads to corneal ulceration, perforation, scar formation, and permanent loss of vision. Prompt corneal healing is important for maintaining corneal epithelial integrity and preserving vision.

Natural epithelial wound healing appears to depend on a complex interaction of various cellular components that cooperate through a network of interactive, signalling molecules. A number of these molecules, known as growth factors, play important roles in corneal wound healing. Epidermal growth factor (EGF), keratinocyte growth factor and platelet-derived growth factor (PDGF) are some of the growth factors known to stimulate corneal wound healing. Interleukin (IL)-1α and IL-6 have also been found to be strongly induced early after corneal alkali burn by the regenerating epithelium, suggesting that they may play a role in regenerating the corneal epithelium.

Lactoferrin is an 80-kDa glycoprotein, the three dimensional structure of which has been defined by X-ray crystallographic analysis. The protein is composed of a single polypeptide chain, which is folded into two globular domains. These domains are termed the N- and C-lobes, which correspond to the amino- (N-lobe) and carboxy (C-lobe) terminal halves of the protein. Each lobe contains one iron-binding site. Lactoferrin has a number of functions, including inflammation reduction, immune response modulation and antibacterial activity. It is a protein found in many species and accordingly reflects some inter-species sequence variation.

Takayama et al (The bovine lactoferrin region responsible for promoting the collagen gel contractile activity of human fibroblasts, Biochem Biophys Res Commun 2002; 299: 813-817) examines the ability of the N- and C-lobes of bovine lactoferrin to promote the contraction of collagen gels by human fibroblasts.

U.S. Pat. No. 7,524,814 relates to a composition comprising whole lactoferrin or an N-terminal lactoferrin variant, in which at least the N-terminal glycine residue is truncated or substituted for use as a treatment for wound healing.

There remains a need for a non-irritating composition that can stimulate corneal epithelial wound repair by means of a practical dosage, i.e. one that is sufficiently potent per unit of mass.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

The present invention relates to a method of treating corneal wounds, which comprises administering to a subject in need thereof a pharmaceutical composition comprising an effective amount of a polypeptide or peptidomimetic comprising the C-lobe of lactoferrin, or functionally active fragments or variants thereof.

The present invention also relates to a pharmaceutical composition comprising an effective amount of a polypeptide or peptidomimetic consisting essentially of the C-lobe of lactoferrin, or functionally active fragments or variants thereof. In one embodiment, the lactoferrin is bovine lactoferrin.

In one embodiment, the peptide or peptidomimetic consists of, or consists essentially of, the C-lobe of lactoferrin. In this specification, “consists essentially of” means, in respect of a peptide or peptidomimetics, an amino acid sequence of any length having substantially the same activity as the C-lobe of bovine lactoferrin as assayed by the method described below and which is at least 60% identical to the sequence of that C-lobe. As illustrated below, the N-lobe and whole lactoferrin have different activity to the C-lobe and therefore a peptide or peptidomimetic that “consists essentially of” the C-lobe of lactoferrin does not include whole lactoferrin. Conveniently, determining whether an amino acid sequence has substantially the same activity as the C-lobe of bovine lactoferrin can be routinely assayed by the cell proliferation and/or migration assays described below.

In one embodiment, the C-lobe is obtained by proteolysis of whole lactoferrin. Preferably, the protease is trypsin. In one embodiment, the lactoferrin is bovine lactoferrin. Optionally it is obtained from cows' milk.

In another embodiment, the subject is a human patient. In one embodiment, the subject has, or is suspected of having, a corneal epithelial wound or injury. This may be separate from or in addition to another injury or injuries. In a further embodiment, the corneal wound is an epithelial corneal wound. In yet a further embodiment, the epithelial corneal wound is an alkali-induced wound.

The present invention also relates to pharmaceutical compositions containing the C-lobe of lactoferrin, or functionally active fragments or variants thereof. In one embodiment, the pharmaceutical composition is in a form suitable for administration to the eye. Preferably, the pharmaceutical composition is an aqueous solution. The pharmaceutical composition is administered topically.

The present invention also relates to a method of treating a corneal wound comprising administration of a therapeutically effective amount of a polypeptide or peptidomimetic comprising the C-lobe of lactoferrin, or functionally active fragments or variants thereof.

The present invention also relates to the use of a therapeutically effective amount of a polypeptide or peptidomimetic comprising the C-lobe of lactoferrin, or functionally active fragments or variants thereof, for the treatment of corneal wounds.

The present invention also relates to the use of a therapeutically effective amount of a polypeptide or peptidomimetic comprising the C-lobe of lactoferrin, or functionally active fragments or variants thereof, for the manufacture of a medicament for the treatment of corneal wounds.

In one embodiment, the invention provides a peptide or peptidomimetic comprising the C-lobe of lactoferrin, or functionally active fragments or variants thereof, when used in a method of treating corneal wounds.

In one embodiment, the invention provides a pharmaceutical composition for treatment of a corneal wound comprising as an active ingredient a polypeptide or peptidomimetic consisting essentially of the C-lobe of lactoferrin, or functionally active fragments or variants thereof. In another embodiment, the invention provides a pharmaceutical composition for treating a corneal wound comprising a polypeptide or peptidomimetic consisting essentially of the C-lobe of lactoferrin, or functionally active fragments or variants thereof as a main ingredient.

The present invention also relates to a method of treating a corneal wound comprising administration of a therapeutically effective amount of a polypeptide or peptidomimetic consisting essentially of the C-lobe of lactoferrin, or functionally active fragments or variants thereof.

The present invention also relates to the use of a therapeutically effective amount of a polypeptide or peptidomimetic consisting essentially of the C-lobe of lactoferrin, or functionally active fragments or variants thereof, for the treatment of corneal wounds. The invention also includes use of this polypeptide or peptidomimetic for the manufacture of a medicament for the treatment of corneal wounds.

The present invention also relates to a method of treating a corneal wound comprising the steps of:
identifying a subject having a corneal wound; and
administering a pharmaceutical composition comprising an effective amount of a polypeptide or peptidomimetic consisting essentially of the C-lobe of lactoferrin, or functionally active fragments or variants thereof, or
administering a therapeutically effective amount of a polypeptide or peptidomimetic consisting essentially of the C-lobe of lactoferrin, or functionally active fragments or variants thereof.

The present invention also relates to a method of accelerating closure of a corneal wound comprising administering to a subject in need thereof:
a pharmaceutical composition comprising an effective amount of a polypeptide or peptidomimetic consisting essentially of the C-lobe of lactoferrin, or functionally active fragments or variants thereof; or
a therapeutically effective amount of a polypeptide or peptidomimetic consisting essentially of the C-lobe of lactoferrin, or functionally active fragments or variants thereof.

In other embodiments there is provided a kit for use in a method of the invention mentioned above, the kit including:
a container holding a peptide, peptidomimetic or pharmaceutical composition of the invention; and
a label or package insert with written instructions for use. Preferably the written instructions describe use of the kit in a method or use of the invention.

In other embodiments there is provided a kit when used in a method of the invention mentioned above, the kit including:
a container holding a peptide, peptidomimetic or pharmaceutical composition of the invention; and
a label or package insert with written instructions for use. Preferably the written instructions describe use of the kit in a method or use of the invention.

In certain embodiments the kit may contain one or more further active principles or ingredients for treatment of a corneal wound.

### BRIEF DESCRIPTION OF THE DRAWINGS/FIGURES

FIG. 1. SEQ ID NO. 1 (publicly available from the Swiss-Prot database under accession number P24627-1, sequence version 2).

FIG. 2. Basic corneal anatomy (stained with hematoxylin and eosin) showing the epithelium, which is the anterior most layer forming the external surface of the cornea.

FIG. 3. Relative closure of alkali-induced HCLE wounds after 24 hours incubation with 12.8 μM bovine lactoferrin: native (BLF); iron free (a-BLF); iron saturated (h-BLF); deglycosylated with TFMS (BLF TFMS); exposed to zwitterionic detergent 2% CHAPS (BLF CHAPS); exposed to chaotrope 6 M Gdn-HCl (BLF Gdn-HCl); reduced and alkylated; and LFcin B peptide compared to BSA control. Data represents mean±SD (n=8). *No statistically significant difference compared with native BLF (p>0.1). ^{#}Statistically significant decrease compared with native BLF (p<0.001).

FIG. 4. Chemical deglycosylation of BLF was confirmed by 7.5% SDS-PAGE under non-reducing conditions and stained with Coomassie R-250. (A) native BLF; (B) BLF incubated for 30 minutes with TMSF.

FIG. 5. Fractions from serine protease affinity column: (A) BLF injected onto column; (B) protein standard; (C) unbound fraction; and (D) eluted fraction. Visualised on 12% SDS-PAGE under reducing conditions and stained with Coomassie R-250.

FIG. 6. Rate of hydrolysis of the serine protease substrate 30 μM Z-Phe-Arg-AMC by 0.1 μM of p-BLF, BLF, C-lobe, np-BLF and BLF treated with 1 μM PMSF. Data represents mean±SD. (n=3 for p-BLF, n=6 for BLF, N-lobe, C-lobe, np-BLF and BLF). *Statistically significant difference compared with p-BLF (p<0.005). ^{#}Statistically significant difference compared with native BLF (p<0.05).

FIG. 7. Closure of alkali-induced HCLE wounds in the presence of 12.6 μM and 252 μM native BLF separated into non-proteolytic (np-BLF) and proteolytic (p-BLF) fractions, with and without serine protease inhibition by 1 mM PMSF. Data represents mean±SD (n=8). *Statistically significant difference compared to PMSF treated (p<0.001). ^{#}Statistically significant difference compared to PMSF treated (p<0.005) ̂Statistically significant difference compared to 1/20^{th} concentration (p<0.001).

FIG. 8. Fractions from the tryptic digestion and purification of BLF N-lobe and C-lobe: (A) Protein standard; (B) tryptic digest of BLF for 4 hours; (C) C-lobe purified from “B” by cation exchange and size exclusion chromatography; (D) BLF; (E) tryptic digest of BLF for 0.5 hour; (F, G, H) BLF, partially digested C-lobe, and N-lobe, respectively, isolated peaks from size exclusion chromatography of “E”. Visualised on 12% SDS-PAGE under reducing conditions and stained with Coomassie R-250.

FIG. 9. Closure of alkali-induced HCLE wounds treated with native BLF, BLF N-lobe, BLF C-lobe, and BSA at 1.28, 6.4, 12.8, 64 and 128 μM concentrations. Data represents mean±SD (n=8). *Statistically significant increase compared to equimolar native BLF (p<0.05) and BLF N-lobe (p<0.001). ^{#}Statistically significant decrease compared to equimolar native BLF (p<0.005) ̂Statistically significant decrease compared to equimolar BSA (p<0.05).

FIG. 10. Closure of debridement wounds in guinea pig eyes treated with 64 μM BLF, N-Lobe, C-lobe, or PBS (Vehicle) expressed as average wound diameter±standard deviation. Dosing with 25 μL every 3 hours for the first 24 hours and then 3 times a day until wound closure. ̂C-Lobe wounds smaller than N-Lobe treated wound (p<0.04) #C-Lobe wounds smaller than PBS treated wounds (p<0.005) *C-Lobe wounds smaller than BLF treated wounds (p=0.02).

FIG. 11. Closure of alkali wounds in guinea pig eyes treated with 64 μM BLF, N-Lobe, C-lobe, or PBS (Vehicle) expressed as average wound diameter±standard deviation. Dosing with 25 μL every 1 hour for the first 8 hours and then 3 times a day until wound closure. #C-Lobe wounds significantly smaller than Vehicle treated wounds (p=0.013).

FIG. 12. Proliferation of Human Corneolimbal Epithelial cells in Medium (M) supplemented with either Bovine Serum Albumin (BSA), Bovine Lactoferrin (BLF), N-Lobe, or C-Lobe at concentrations of 1.28, 6.4, 12.8, 64, and 128 μM. Measured by CyQuant after 0, 8, 16 and 24 hours incubation. n=8 for all groups. #Less proliferation than equimolar BSA (p<0.001) *Greater proliferation than equimolar BSA (p<0.05).

FIG. 13. Wound closure by migration of Human Corneolimbal Epithelial Cells while proliferation is inhibited with 1 mM hydroxyurea in Medium (M) supplemented with either Bovine Serum Albumin (BSA), Bovine Lactoferrin (BLF), N-Lobe, or C-Lobe at concentrations of 1.28, 6.4, 12.8, 64, and 128 μM. n=8 for all groups. Measurements taken at 0, 8, 16 and 24 hours after migration barrier removed *Greater migration than equimolar BSA (p<0.05). #Less migration than equimolar BSA (p<0.001).

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

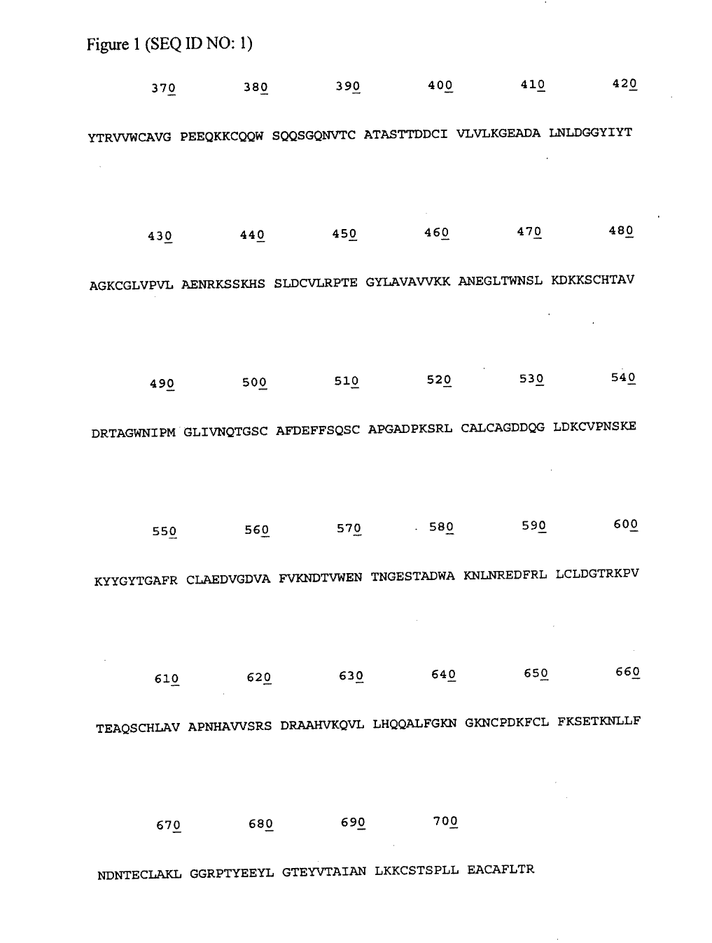

The term “C-lobe of lactoferrin” refers to the C-terminal lobe of lactoferrin. As discussed previously, the protein is composed of a single polypeptide chain, which is folded into two globular domains. These domains are termed the N- and C-lobes, which correspond to the amino- (N-lobe) and carboxy (C-lobe) terminal halves of the protein. Each lobe contains one iron-binding site. It has been shown that the lactoferrin protein is approximately 690 amino acids long, with the C-lobe corresponding to the amino acid sequence from approximately amino acid 364 (at least for bovine lactoferrin) to the C-terminal end (e.g. amino acid 690). The N-terminal end of the C-lobe may be located at amino acid 364, or within two to three amino acids of that position (e.g. amino acid 361 to amino acid 366). In one embodiment, the amino acid sequence of the C-lobe is that given in FIG. 1 (defined as SEQ ID NO 1). The invention extends to all published sequences of lactoferrin and the C-lobe sequence they contain.

In a preferred embodiment, the C-lobe is derived from bovine lactoferrin and has the sequence according to SEQ ID NO: 1. As stated herein, the present invention also includes variants, for example species variants or polymorphic variants, including an amino acid sequence as described below where any one or more of the 0 amino acids in parenthesis replace the amino acid preceding it.

YTRVVWCAVGPEEQKKCQQWSQQSGQNVTCATASTTDDCIVLVLKGEADALNLDGGYI(V)
YTAGKCGLVPVLAENRKS(T)SKH(Y)SSLDCVLRPTEGYLAVAVVK(R)KANEGLTWNS
LKDKKSCHTAVDRTAGWNIPMGLIVNQTGSCAFDEFFSQSCAPGA(R)DPKSRLCALCAGD
DQGLDKCVPNSKEKYYGYTGAFRCLAEDVGDVAFVKNDTVWENTNGESTADWAKNLN
REDFRLLCLDGTRKPVTEAQSCHLAVAPNHAVVSRSDRAAHVKQVLLH(R)QQALFGKNG
KNCPDKFCLFKSETKNLLFNDNTECLAKLGGRPTYEEYLGTEYVTAIANLKKCSTSPLLEA
CAFLTR

The term “polypeptide” or “polypeptide chain” refers to a polymer of amino acids, usually linked together by amide bonds. A functionally-active polymer of amino acids is generally referred to as a “protein”.

There are a number of isoforms of lactoferrin and therefore the exact number of amino acids that make up the lactoferrin protein will vary. Accordingly, the exact location of the C-lobe within the protein will also vary. The present invention is intended to cover all functionally active fragments and variants of the C-lobe that exhibit the same activity as assayed by the method described below. This also includes apo- and holo-forms of the C-lobe, post-translationally modified forms, as well as glycosylated or de-glycosylated derivatives. The C-lobe may optionally include the interlobe region, or part thereof, which occurs between the C-lobe and N-lobe in whole lactoferrin. The interlobe region may have a sequence of any isoform or species variant of lactoferrin.

The term “functionally active” in relation to a fragment or variant of the polypeptide sequence of the C-lobe of lactoferrin means that the fragment or variant (such as an analogue, derivative or mutant) that is capable of healing corneal wounds, by, for example, being applied to the wound to be treated as assayed by the method described below. Such variants include naturally occurring variants and non-naturally occurring variants. Additions, deletions, substitutions and derivatizations of one or more of the amino acids are contemplated so long as the modifications do not result in loss of functional activity of the fragment or variant. A functionally active fragment can be easily determined by shortening the amino acid sequence, for example using an exopeptidase, or by synthesizing amino acid sequences of shorter length, and then testing for any wound healing activity such as by the methods illustrated in the examples below.

Where non-natural variations occur, the fragment may be called a peptidomimetic, which are also within the scope of the invention. For example, synthetic amino acids and their analogues may be substituted for one or more of the native amino acids providing wound healing activity as assayed in the method below.

A “peptidomimetic” is a synthetic chemical compound that has substantially the same structure and/or functional characteristics of a peptide of the invention, the latter being described further herein. Typically, a peptidomimetic has the same or similar structure as a peptide of the invention, for example the same or similar sequence of a C-lobe of lactoferrin. A peptidomimetic generally contains at least one residue that is not naturally synthesised. Non-natural components of peptidomimetic compounds may be according to one or more of: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

Peptidomimetics can be synthesized using a variety of procedures and methodologies described in the scientific and patent literatures (e.g., Organic Syntheses Collective Volumes, Gilman et al. (eds) John Wiley & Sons, Inc., NY; al-Obeidi; Mol Biotechnol 1998; 9: 205-223; Hruby Curr Opin Chem Biol 1997; 1: 114-119; Ostergaard Mol Divers 1997; 3:17-27; Ostresh Methods Enzymol 1996; 267: 220-234.

Preferably, the functionally active fragment is 30, 40, 50, 60, 70, 80, 90 or greater amino acids in length. Preferably, the functionally active fragment or variant has at least approximately 60% identity to the relevant part of SEQ ID NO 1 to which the fragment or variant corresponds, more preferably at least approximately 65%, 70%, 75%, 80% or 85% identity, even more preferably 90% identity, even more preferably at least approximately 95%, 96%, 97%, 98%, 99% or 100% identity. The functionally active fragment or variant may correspond to, or have identity with, a contiguous sequence of amino acids from the C-lobe of lactoferrin, however it is also contemplated that a functionally active fragment corresponds to, or has identity with, sequences of amino acids that are clustered spatially in the three dimensional structure of the C-lobe of lactoferrin.

Such functionally active fragments and variants include, for example, those having conservative amino acid substitutions. Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=(X/Y)×100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of 13 to A.

In calculating percent identity, exact matches are counted. The determination of percent identity between two sequences can be accomplished using a mathematical algorithm. A non-limiting example of a mathematical algorithm utilized for the comparison of two sequences is the algorithm of Karlin and Altschul Proc Natl Acad Sci 1990 USA; 87: 2264, modified as in Karlin and Altschul Proc Natl Acad Sci USA 1993; 90: 5873-5877. Such an algorithm is incorporated into the BLASTN and BLASTX programs of Altschul et al. J MoI Biol 1990; 215: 403. To obtain gapped alignments for comparison purposes, Gapped BLAST (in BLAST 2.0) can be utilized as described in Altschul et al. (1997) Nucleic Acids Res 25: 3389. Alternatively, PSI-Blast can be used to perform an iterated search that detects distant relationships between molecules. See Altschul et al. (1997) supra. In one preferred embodiment, utilizing BLAST, Gapped BLAST, and PSI-Blast programs, the default parameters of the respective programs (e.g., BLASTX and BLASTN) are used. Alignment may also be performed manually by inspection. Another non-limiting example of a mathematical algorithm utilized for the comparison of sequences is the ClustalW algorithm (Higgins et al. Nucleic Acids. Res 1994; 22: 4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting example of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (CABIOS 1988; 4: 11-17). Such an algorithm is incorporated into the ALIGN program (version 2.0), which is part of the GCG Wisconsin Genetics Software Package, Version 10 (available from Accelrys, Inc., 9685 Scranton Rd., San Diego, Calif., USA). In one preferred embodiment, utilizing the ALIGN program for comparing amino acid sequences, a PAM 120 weight residue table, a gap length penalty of 12, and a gap penalty of 4 is used when assessing percentage identity.

The term “conservative amino acid substitutions” refers to the substitution of an amino acid by another one of the same class, the classes being as follows:
Non-polar: Ala, Val, Leu, Ile, Pro, Met Phe, Trp
Uncharged polar: Gly, Ser, Thr, Cys, Tyr, Asn, Gln
Acidic: Asp, Glu
Basic: Lys, Arg, His

Other conservative amino acid substitutions may also be made as follows:
Aromatic: Phe, Tyr, His
Proton Donor: Asn, Gln, Lys, Arg, His, Trp
Proton Acceptor Glu, Asp, Thr, Ser, Tyr, Asn, Gln

The terms “treating” or “treatment” refer to administering to a subject a therapeutically effective amount of a composition comprising the peptide or peptidomimetics (such as the C-lobe of lactoferrin), such that the subject has an improvement in the condition to be treated (e.g. a corneal wound). It will be recognised that the treatment may improve the condition, but may not provide a complete cure for the condition. The pharmaceutical composition may comprise the C-lobe of lactoferrin, or one or more functionally active fragments or variants thereof.

The term “subject” refers to any animal to which a composition containing the C-lobe of lactoferrin is administered. In a preferred embodiment, the subject is a human patient who is suffering from a wound. The wound is preferably a corneal wound, and in one embodiment a corneal epithelial wound. Although the invention finds application in humans, the invention is also useful for veterinary purposes. The invention is useful for the treatment of wounds, as described herein, in domestic animals such as cattle, sheep, horses and poultry; companion animals such as cats and dogs; and zoo animals.

The terms “therapeutically effective amount” or “effective amount” refer to an amount of the peptide or peptidomimetic that results in an improvement or remediation of one or more of the symptoms of the disease or condition.

The term “wound” refers to an injury, such as an ulcer or lesion, as a result of a disease or disorder, or as a result of an accident, incident or surgical procedure (e.g. LASIK or PRK). For example, the wound may be an abrasion, which is caused by contact of the cornea with foreign bodies (e.g. sand) or contact lenses. The wound may be a corneal wound (including specifically a corneal epithelial wound, together with or without other wound or injury) that is a result of an alkali injury, i.e. an alkali-induced wound, or any other chemical burn. The ulcer may be of infectious, inflammatory or autoimmune origin. The lesion may be a non-healing corneal lesion. The wound may also be a result of a dry eye condition.

The term “pharmaceutical composition” refers to a composition comprising the peptide or peptidomimetics (such as the C-lobe of lactoferrin), which is dispersed in a pharmaceutically acceptable carrier. The pharmaceutical composition may comprise the C-lobe of lactoferrin, or one or more functionally active fragments or variants thereof. The composition may further include one or more additional excipients, such as diluents, emulsifiers, buffers, stabilizing agents, binders, fillers, and the like. Optionally it may also include an effective amount of other pharmaceutically active components. For example, an antibiotic could also be included, such as a member of the quinolone family or a combination of aminoglycoside and a beta-lactam. Other antibiotics including, but not limited to, chloramphenicol, tetracyclines and macrolides could also be used.

Further, the composition may include one or more anti-inflammatory agents that may be steroidal or non-steroidal anti-inflammatory agents.

The pharmaceutical composition of the invention may also contain only (i.e. consist essentially of) the C-lobe of lactoferrin. Alternatively, the invention includes a pharmaceutical composition that contains a greater concentration of a peptide or peptidomimetic consisting essentially of the C-lobe of lactoferrin, or functionally active fragments or variants thereof, than any other peptide, peptidomimetic and/or other active ingredient.

As used herein, except where the context requires otherwise, the term “comprise” and variations of the term, such as “comprising”, “comprises” and “comprised”, are not intended to exclude further additives, components, integers or steps.

The present invention treats corneal wounds, and involves administering to a subject a pharmaceutical composition comprising an effective amount of a peptide or peptidomimetic such as the C-lobe of lactoferrin, or functionally active fragments or variants thereof. The present invention is particularly concerned with the treatment of corneal wounds. In particular, the types of corneal wounds contemplated by the present invention are epithelial corneal wounds. The wounds may be the result of, for example, chemical injuries, such as those caused by exposure of the eye to alkali agents (i.e. alkali-induced wounds) or surgical alcohol debridement. Alkali-induced wounds can occur, for example, by accidental exposure of the eye to alkali liquids, fertilizers, plaster and cement powders, household cleaning products (particularly those containing ammonia), drain cleaners, oven cleaners and the like. The invention also assists to minimise entry of pathogens into the cornea.

Alkali exposure causes epithelial cell death, denaturation of stromal collagen and imperils the cornea and internal eye to invasion by foreign bodies and pathological agents. Alkali-induced wounds are characterized by a heightened inflammatory response and impeded wound healing, which prolongs the risk period in which sight-threatening secondary complications (e.g. microbial infections) can occur. Severe injuries can also result in recurring epithelial ulcerations, chronic stromal ulcers, profound stromal neovascularization, conjunctival overgrowth, or even corneal perforation.

Other corneal wounds that may be treated with a peptide or peptidomimetic of the invention or by a method or use of the invention are wounds arising from debridement, abrasions, scratches or any other abrasive injury. These wounds are generally considered to be non-inflammatory wounds.

The applicants have found that the C-lobe of lactoferrin is able to increase wound closure rates more potently than either the N-lobe or native (i.e. whole) lactoferrin.

The promotion of healing of a different part of the cornea, corneal stromal wound healing, by native lactoferrin has been previously attributed to its stimulation of fibroblast proliferation (which results in the synthesis of extracellular matrix). In contrast, the present invention is concerned with the treatment of wounds of the corneal epithelium, which does not contain fibroblasts. Without wishing to be bound by any theory or mode of action, it is proposed by the applicants that the C-lobe of lactoferrin increases rates of epithelial wound closure by promoting the migration of epithelial cells across the ocular surface and up-regulating the expression of various cytokines and growth factors (e.g. IL-6 and PDGF).

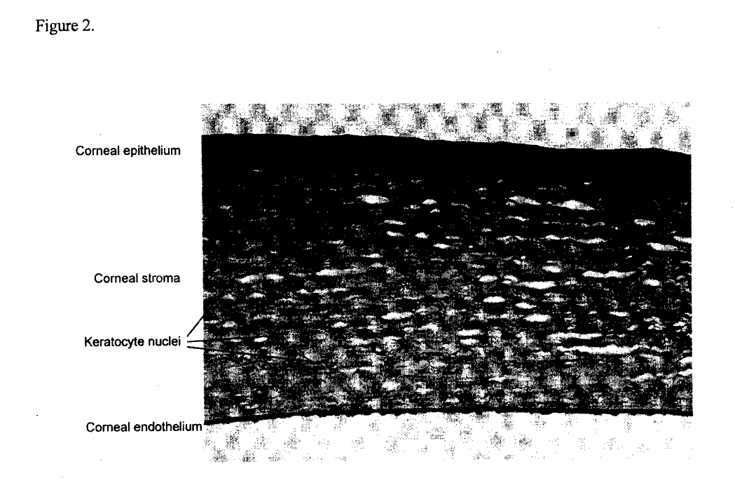

FIG. 2 shows the basic corneal anatomy. The epithelium is the anterior most layer forming the external surface of the cornea. This layer is predominantly cellular (composed of keratinocytes). The stroma is underneath the epithelium and contains the keratocytes. It is mostly composed of collagen. The keratocytes form a loosely connected network between collagen layers joined by very fine branches and account for about 10% of the stroma. The migration of corneal epithelial cells (keratinocytes) occurs over a provisional matrix of fibronectin, an adhesive extracellular glycoprotein, which appears at the exposed surface of the stroma at corneal epithelial wound sites. It has been shown that the expression of fibronectin increases after injury and that certain growth factors are able to enhance the effects of fibronectin on cell migration. In the case of epithelial wound healing, it is proposed that the up-regulation of these growth factors by native lactoferrin can be attributed to its interaction with various receptors, such as those involved in wound healing and PDGF-signalling pathways.

Again, without wishing to be bound by any theory or mode of action, the C-lobe's increased efficacy compared to the N-lobe and native lactoferrin may be due to steric factors, greater substrate affinity or an inhibitory effect from the N-lobe. For example, liberating the C-lobe from the unnecessary 40 kDa of the N-lobe could reduce steric interference of the peptides at a particular target binding site, thereby promoting would healing. Alternatively, attraction of the cationic arginines near the N-terminal of lactoferrin to ubiquitous anionic substrates (e.g. sulphated aminoglycans), would reduce the lactoferrin that is available to bind the target for promotion of wound healing. Lastly, an activity (e.g. proteolytic activity) that is mildly antagonistic to wound closure may be present on N-lobe peptides.

The present invention also relates to a method of accelerating closure of a corneal wound comprising administering to a subject in need thereof a pharmaceutical composition comprising an effective amount of a polypeptide or peptidomimetic comprising the C-lobe of lactoferrin, or functionally active fragments or variants thereof or a therapeutically effective amount of a polypeptide or peptidomimetic comprising the C-lobe of lactoferrin, or functionally active fragments or variants thereof. The closure of a wound treated by a peptide or peptidomimetic of the invention is accelerated in comparison to an untreated wound and/or a wound treated by whole lactoferrin. Accelerated closure of a corneal wound is advantageous to prevent additional wounding to the cornea and/or to minimise the risk of infection or ulceration. In addition, accelerating wound closure results in a rapid resolution of visual function.

It will be understood by a person skilled in the art that the C-lobe of lactoferrin can be obtained by any suitable method known to the skilled person including, but not limited to: recombinant techniques, synthesis de novo using genetic engineering and/or chemical synthesis techniques; isolation from natural sources (e.g. mammalian milk), purification and proteolysis; and purchase from commercial sources. In this way, the C-lobe may be purified, isolated, recombinant or synthetic.

In a preferred embodiment, the C-lobe is obtained by proteolysis of naturally sourced, recombinant or commercially available lactoferrin into its N- and C-lobes. Preferably, the protease used is trypsin. The N- and C-lobes can then be separated from each other using any number of techniques known to the skilled person e.g. chromatography. Cation exchange and size exclusion chromatography are suitable methods.

The concentration of the peptide or peptidomimetics, such as the C-lobe, present in the pharmaceutical compositions of the present invention may be, for example, between 10 to 70 μM.

The pharmaceutical composition of the present invention may be an ophthalmic composition, which is a composition suitable for administration or application to the eye. Examples of ophthalmic compositions according to the invention are suspensions, ointments, sustained release formulations (including when loaded into a contact lens or other biomaterial), gels or solutions suitable for application as an eye drop. Preferably, the pharmaceutical compositions according to the present invention will be formulated for topical administration or for sustained release delivery. Preferably, the composition of the present invention is in a form suitable for administration to the eye. Aqueous solutions are generally preferred, based on ease of formulation, as well as a subject's ability to easily administer such compositions by means of instilling one to two drops of the solutions in the affected eyes. However, the compositions may also be suspensions, viscous or semi-viscous gels, or other types of solid or semi-solid compositions, or those appropriate for sustained release. The pharmaceutical composition may be an ocular lubricant, such as an artificial tear formulation, or contact lens solution.

Any of a variety of carriers may be used in the compositions of the present invention including water, mixtures of water and water-miscible solvents, such as C₁ to C₇ alkanols, vegetable oils or mineral oils comprising from 0.5 to 5% non-toxic water-soluble polymers, gelling products, such as gelatin, alginates, pectins, tragacanth, karaya gum, xanthan gum, carrageenin, agar and acacia, and their derivatives, starch derivatives, such as starch acetate and hydroxypropyl starch, cellulose and its derivatives and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers, naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan mono-oleate.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

The composition according to the present invention may comprise at least one gelling agent. Gelling agents suitable for use in pharmaceutical compositions are well known to those of ordinary skill in the art and include, for example, xanthan gum and its derivatives, carbomer and its derivatives, acrylate based copolymers and cross polymers, sodium polyacrylate and its derivatives, cellulose and its derivatives, and starch and agar and their derivatives. The selection of the gelling agent according to the present invention is important in providing a clear gel. The amount of gelling agent added to the composition may be readily determined by one of ordinary skill in the art with a minimum of experimentation, and will depend upon factors known to those skilled in the art, such as the properties of the gelling agent and the desired properties of the pharmaceutical composition.

Additional ingredients that may be included in the pharmaceutical composition of the invention include tonicity enhancers, preservatives, solubilizers, stabilizers, non-toxic excipients, demulcents, sequestering agents, pH adjusting agents, co-solvents and viscosity building agents. For the adjustment of the pH, preferably to a physiological pH, buffers may especially be useful. The pH of the present solutions should be maintained within the range of between 4 to 8, preferably 6 to 7.5. It will be understood by a person of ordinary skill in the art that any pH that is compatible with the ocular surface is suitable. Suitable buffers may be added, such as boric acid, sodium borate, potassium citrate, citric acid, sodium bicarbonate, TRIS, disodium edetate (EDTA) and various mixed phosphate buffers (including combinations of Na₂HPO₄, NaH2PO₄ and KH₂PO₄) and mixtures thereof. Generally, buffers will be used in concentrations ranging from about 0.05 to 0.5 M.

Tonicity is adjusted if needed typically by tonicity enhancing agents. Such agents may, for example, be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids.

In certain embodiments, the compositions of the invention additionally comprise a preservative. A preservative may typically be selected from a quaternary ammonium compound such as benzalkonium chloride (N-benzyl-N—(C₈-C₁₈ alkyl)-N,N-dimethylammonium chloride), benzoxonium chloride or the like. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, sodium benzoate, salicylic acid, alcohols, such as, for example, chlorobutanol, benzyl alcohol or phenyl ethanol, guanidine derivatives, such as, for example, chlorohexidine or polyhexamethylene biguanide, sodium perborate, Germal® π or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In other embodiments, the compositions of this invention do not include a preservative. Such formulations would be particularly useful for subjects who wear contact lenses.

The composition of the invention may additionally require the presence of a solubilizer, in particular if the active or the inactive ingredients tend to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient.

The compositions may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and it will be understood by a person of ordinary skill in the art what types and amounts of excipients and other additives may be present in a composition such that the composition is compatible with the eye. Other compounds may also be added to the compositions of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Exemplary ophthalmic solutions of the invention include a peptide or peptidomimetic of the invention, sodium chloride, disodium maleate, benzalkonium chloride, sodium hydroxide, hydrochloric acid, sterile purified water and the solution having a physiological pH of about 7.45 or a pH within the ocular comfort range. For maximum comfort, an ophthalmic solution should have the same pH as the lacrimal fluid or the pH of the solution should lie within the ocular comfort range, i.e. between pH 6.6 to 7.8. Alternatively, the solution may include a peptide or peptidomimetic of the invention, sodium chloride, sodium dihydrogen phosphate dihydrate, benzalkonium chloride, sodium hydroxide, hydrochloric acid, sterile purified water and the solution having a pH as discussed above.

An exemplary ophthalmic solution is:

| | |
|---|---|
| Peptide or peptidomimetic of the invention | 0.3%-0.5% (w/v) |
| Sodium chloride | 0.9% (w/v) |
| Sodium dihydrogen phosphate dihydrate | 0.08% (w/v) |
| Benzalkonium chloride | 0.005% (w/v) |
| Sterile water | q.s. |

where the pH of the solution is adjusted to a physiological pH or a pH within the ocular comfort range with any biocompatible acid and/or alkali, such as sodium hydroxide and hydrochloric acid.

The pharmaceutical compositions of the present invention may contain other active ingredients that are effective in the treatment of wounds e.g. growth factors, cleansers and antibiotics. The pharmaceutical composition can also be administered in combination with a treatment such as skin replacement therapy, enzymatic and surgical debridement, wound dressing and compression. Generally, these active ingredients and treatments are provided in a combined amount effective to promote the healing of a wound. This may involve administering the composition of the present invention and the active ingredient/treatment at the same time or at times close enough such that the administiation results in an overlap of the desired effect. Alternatively, the composition of the present invention may precede or follow other treatments. A composition of the invention may be administered during or following an elective surgery, such as LASIK surgery.

The composition may be administered in any way that is deemed suitable by a person of ordinary skill in the art. The pharmaceutical composition may be administered topically.

The composition of the invention may be administered in single or multiple doses and for any length of time until the wound is either completely healed or until the desired level of wound healing has been achieved. The person of ordinary skill in the art will recognise that the dosage amount, dosage regime and length of treatment will depend on factors such as, for example, the wound type, the location of the wound and the health of the subject. In the case of chemical injuries, the treatment required will depend on factors such as the extent of the ocular surface damaged, the degree of intraocular penetration by the chemical agent, and the concentration and nature of the agent involved. In one embodiment, the composition is administered every half hour or hourly, up to, for example, eight times a day.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a peptide, peptidomimetic or pharmaceutical composition which is effective for treating the condition and may have a sterile access port (for example the container may be an intravenous solution bag or a vial having a stopper pierceable by a hypodermic injection needle). The label or package insert indicates that the peptide, peptidomimetic or pharmaceutical composition is used for treating the condition of choice. In one embodiment, the label or package insert includes instructions for use and indicates that the therapeutic composition can be used to treat a corneal wound.

The kit may comprise (a) a peptide, peptidomimetic or pharmaceutical composition; and (b) a second container with a second active principle or ingredient contained therein. The kit in this embodiment of the invention may further comprise a package insert indicating that a peptide, peptidomimetic or pharmaceutical composition and other active principle can be used to treat a corneal wound. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The present invention will now be more fully described with reference to the accompanying examples and drawings. It should be understood, however, that the description following is illustrative only and should not be taken in any way as a restriction on the generality of the invention described above.

### EXAMPLES

The inventors identified the structures of lactoferrin that promote human corneal epithelial wound healing using an alkali-induced wound model.

In summary, the BLF lobes were separated by limited tryptic proteolysis and purified using cation exchange and size exclusion chromatography. Isoforms of bovine lactoferrin (BLF) were separated according to their serine protease activity with a benzamidine affinity column and their catalytic activities, and those of the BLF lobes, were quantified by hydrolysis of the synthetic serine protease substrate Z-Phe-Arg-7-amide-4-methyl-coumarin. The promotion of wound healing by these moieties and of BLF (iron-free, iron-bound, deglycosylated, zwitterionic detergent exposed, chaotrope denatured, reduced and alkylated, and lactoferrin B peptides (LFcin B)) were assessed by incubation with confluent monolayers of human corneolimbal epithelial cells wounded with filter paper discs soaked in 0.1 M sodium hydroxide.

BLF endotoxin content was analysed with Limulus amoebocyte lysate assay (QCL-1000; Lonza, Walkersville, Md.) as per the manufacturer's instructions.

Iron-free (apo) bovine lactoferrin (a-BLF) was prepared as described by Masson et al (Metal-combining properties of human lactoferrin (red milk protein). 1. The involvement of bicarbonate in the reaction. Eur J Biochem 1968; 6: 579-584) with modifications. The iron of a 1% solution of BLF (a gift from Dr Andrew Brown, Murray Goulburn Co-operative, Cobram, VIC, Australia) was removed against 0.1 M citric acid in a centrifugal ultrafiltration device (10 kDa cut-off Amicon Ultra; Millipore, Bedford, Mass.) at 4° C. The resulting clear solution was then buffer exchanged to phosphate buffered saline (PBS) and concentrated by ultrafiltration.

Iron-saturated (holo) bovine lactoferrin (h-BLF) was prepared by the addition of the iron complex ferric-nitrilotriacetate (Fe-NTA) by a similar method to Bates et al (The reaction of ferric salts with transferrin. J Biol Chem 1973; 248: 3228-3232). A 1% solution of BLF in 20 mM Tris-HCl buffer pH 7.4 with 5 mM bicarbonate added immediately prior to combination with a 2:1 molar excess of Fe-NTA and incubated for 1 hour. The h-BLF was then buffer exchanged to PBS and concentrated as above.

Iron-saturation of a-BLF was confirmed spectrophotometrically by the ratio of 280 nm to 465 nm absorbance (Structural studies on bovine lactoferrin. J Biol Chem 1970; 245: 4269-4275).

Glycan chains were removed chemically from BLF following the process of Sojar and Bahl (A chemical method for the deglycosylation of proteins. Arch Biochem Biophys 1987; 259: 52-57). BLF in a 10% solution was incubated in anhydrous trifluoromethanesulfonic acid (TFMS; Sigma) on ice for 30 minutes followed by neutralization with 60% pyridine at −20° C. then buffer exchanged to PBS. Progress was monitored by reduction in apparent molecular weight of the BLF bands with sodium dodecyl sulphate-polyacrylamide gel electrophoresis (SDS-PAGE) in 7.5% tris-HCl polyacrylamide gel.

A preparation of reduced and alkylated BLF was prepared as follows. A 1% solution of BLF in 0.6 M Tris-HCl pH 8.5 and 2% (3-((3-cholamidopropyl)dimethylammonio)-1-propanesulfonate (CHAPS; Sigma) with and without 6 M guanidine hydrochloride (Gdn-HCl; Sigma) was reduced by incubation with β-mercaptoethanol (Sigma), in a 50 fold molar excess to the disulphide bonds, for 4 hours. Alkylation was by addition of freshly prepared iodoacetamide (Sigma) to a concentration slightly below the reducing agent (e.g. 6 mM). The solution was protected from light during the 15 minute incubation before buffer exchange to PBS at 4° C.

### Serine Protease Activity and Isolation

Fractions of BLF with proteolytic activity were purified with a benzamidine serine protease affinity column (GE Healthcare, Uppsala, Sweden) used according to the manufacturer's protocol. Briefly, BLF was loaded onto the column in 50 mM Tris-HCl buffer with 0.5 M NaCl at pH 7.4 and the bound fractions were eluted at pH 2.0 into a collection buffer restoring pH to physiological levels. Irreversible inhibition of BLF proteolytic activity was by addition of 1 mM phenylmethanesulphonyl fluoride (PMSF; Fluka Analytical, Buchs, SG, Switzerland) at a 10:1 molar excess subsequently removed by buffer exchange. Quantification of BLF proteolytic activity was adapted from Massucci et al (Proteolytic activity of bovine lactoferrin. Biometals 2004; 17: 249-255). Serine protease activity measurements were made with the substrate N-α-benzyloxycarbonyl-phenylalanine-arginine-7-amido-4-methyl-coumarin (Z-Phe-Arg-AMC; Sigma-Aldrich, St Louis, Mo.) at concentrations from 3 to 300 μM in 20 mM phosphate buffer pH 7.0 with 100 mM NaCl at 25° C. Cleavage of the peptide and release of the AMC group by 0.1 μM of BLF was monitored spectrofluorimetrically by 465 nm emission and 360 nm excitation wavelengths to calculate the initial reaction velocity. The kinetic parameters Kₘ and k_{cat} were extrapolated by linear regression of the Lineweaver-Burk plot. Comparisons of the reaction rates of BLF serine protease affinity column fractions, BLF lobes and serine protease inhibited BLF were made using 30 μM Z-Phe-Arg-AMC.

### BLF Lobe Purification

Separation of BLF into N-lobe and C-lobe fragments was modified from Legrand (Characterization and localization of an iron-binding 18-kDa glycopeptide isolated from the N-terminal half of human lactotransferrin. Biochim Biophys Acta 1984; 787: 90-96). BLF in 0.1 M Tris-HCl buffer pH 8.2 containing 25 mM CaCl₂ was digested with 25 TAME units of immobilised trypsin (Pierce, Rockford, Ill.) per mg substrate at 37° C. with moderate agitation (one TAME unit hydrolyses 1 μmole of p-toluenesulphonyl-L-arginine methyl ester (TAME) per minute at 25° C. and pH 8.2, in the presence of 10 mM calcium). Incubation times of 0.5 and 4 hours were used to maximise yield of N-lobe and C-lobe respectively. The reaction was terminated by centrifugal separation of trypsin gel from the sample as per manufacturer's directions.

The lobes were purified by cation exchange chromatography using a Mono S 5/50 GL column (GE Healthcare) equilibrated in 50 mM HEPES pH 8.0. Elution was carried out by a linear gradient up to 1 M NaCl in the same buffer. The isolated peaks were applied to a size exclusion column Bio-Gel P-60 26/1000 (Bio-Rad Laboratories, Hercules, Calif.) in 10% acetic acid (Legrand, referenced above) and 150 mM NaCl at 0.4 mL/min. Visualisation of BLF and fragments by SDS-PAGE with the Laemmli system (Cleavage of structural proteins during the assembly of the head of bacteriophage T4. Nature 1970; 227: 680-685) on 12% Tris-HCl gels stained with Coomassie Blue R-250 (Bio-Rad Laboratories). Apparent molecular weight of reduced, heat denatured samples was calculated against protein standard (Precision Plus, Bio-Rad) using 1-D gel analysis software (Quantity One, Bio-Rad).

Identity of BLF fragments by N-terminal sequencing of the first 5 amino acids of polyacrylamide gel bands extracted by passive elution was prepared to verify the fractions collected.

### Cell Culture

Immortalized human corneal-limbal epithelial (HCLE) cells (a gift from Dr Ilene Gipson, Schepens Eye Research Institute, Boston, Mass.) were cultured as previously described (Mucin gene expression in immortalized human corneal-limbal and conjunctival epithelial cell lines. Invest Ophthalmol Vis Sci 2003; 44: 2496-2506). Briefly, cells were seeded at 2×10⁴/cm onto tissue culture treated plates and maintained in keratinocyte serum-free medium (K-SFM; Invitrogen-Gibco, Grand Island, N.Y.), supplemented with 25 ug/mL bovine pituitary extract, 0.2 ng/mL recombinant epidermal growth factor, and 0.4 mM CaCl₂ at 37° C. in a 5% CO₂ atmosphere. At 50% confluence they were switched to a 1:1 mixture of K-SFM and low-calcium Dulbecco's modified Eagle medium (DMEM)/Ham's F12 (Invitrogen) to achieve confluence.

### HCLE Alkali Burn Wound Healing Model

To determine the effect of BLF derivatives on healing of alkali-induced burns, confluent monolayers of HCLE cells were wounded using filter paper discs soaked in 0.1 M sodium hydroxide. Cells were immediately rinsed by three culture medium (1:1 K-SFM:low Ca²⁺ DMEM/F12) changes to restore pH and remove cellular debris. The wound area was photographed at 50× magnification before and after 24 hours incubation in the treatment solution at 37° C. in 5% CO₂. Areas of wounds were quantified using image analysis software (ImageJ 1.40 g; National Institutes of Health, Bethesda, Md.). Results were expressed as either relative wound closure, this is the reduction in wound area as a multiple of the control, or percentage wound closure, the reduction in wound area compared to initial wound area.

The treatment solutions for the alkali burn wound healing model were prepared by diluting concentrated BLF; apo, hobo, deglycosylated, CHAPS exposed, Gdn-HCl exposed, reduced and alkylated, and LFein B (American Peptide, Vista, Calif.) to 12.8 μM in tissue culture medium (as discussed above). Benzamidine column fractions reconstituted to the concentrations present in native BLF of 12.6 μM and 254 μM with and without PMSF pre-treatment. BLF N-lobe and C-lobe prepared to final concentrations of 1.28, 6.4, 12.8, 64 and 128 μM. Positive and negative controls of equimolar native BLF and bovine serum albumin (BSA; Bovogen Biologicals, Essendon, VIC, Australia) were included, respectively, in each experiment. The LFcin B used was synthesised de novo and corresponds to BLF amino acids 20 to 31.

### Statistical Analysis

For wound healing experiments data summarised as mean±SD of a sample size 8 for each treatment at a concentration. Results of BLF; apo, holo, deglycosylated, CHAPS exposed, Gdn-HCl exposed, reduced and alkylated, LFcin B, N-lobe and C-lobe were assessed to determine differences between the treatments within a concentration using one-way analysis of variance (ANOVA) followed by post hoc multiple comparisons using Bonferroni correction.

Analysis of results for wound healing trials with benzamidine column fractions were analysed as above with an additional comparison made between concentrations. For reaction rate experiments differences between moieties were calculated using one-way ANOVA followed by post hoc multiple comparisons using Games-Howell correction due to the sample size and variance of the groups.

Statistical significance was taken as p<0.05. Analysis was performed using commercial statistical analysis software (SPSS; SPSS Inc., Chicago, Ill.).

### Results

Endotoxin content was found to be less than 4 EU/mg, as determined by the LAL assay, in all BLF used in these experiments.

Iron saturation of BLF did not alter the promotion of wound closure following alkali injury to IICLE monolayers. Spectroscopic analysis indicated iron saturation to be less that 10% for a-BLF and more than 90% for h-BLF. A significant increase in wound closure was found for a-BLF, native

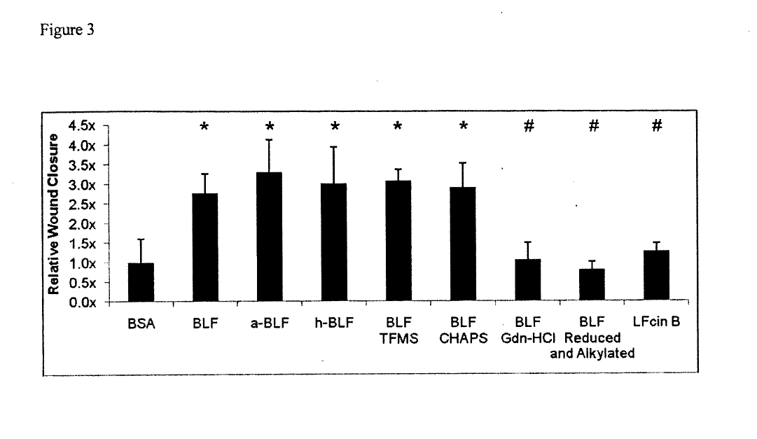

BLF and h-BLF compared to the BSA control (p<0.001; FIG. 3). A 3 fold order of increase in wound closure compared to the BSA control was found for a-BLF, native BLF and h-BLF at 12.8 μM concentrations.

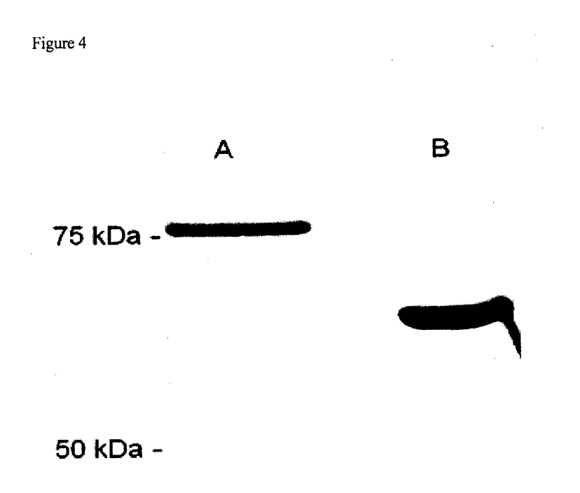

Removal of glycans from BLF did not alter its promotion of wound healing. Chemical deglycosylation was completed after 30 minutes with no further decrease in apparent molecular weight observed by SDS-PAGE (FIG. 4). An equivalent apparent molecular weight change was observed for BLF enzymatically deglycosylated with peptide-N-glycosidase F under denaturing conditions (data not shown). Deglycosylated BLF significantly increased closure of alkali-induced corneal wounds compared to BSA (p<0.001, FIG. 3). This effect was not significantly different from native BLF (p>0.1, FIG. 3).

BLF prepared using a chaotrope, 6 M Gdn-HCl, produced significantly less wound closure compared to native BLF (p<0.001; FIG. 3) while BLF pre-treated with the zwitterionic detergent (2% CHAPS) continued to increase wound healing. Promoting effect of BLF on wound healing was lost following its reduction and alkylation.

In isolation the LFcin B peptide did not promote closure of alkali-induced wounds in HCLE cells. Less wound healing was observed for LFcin B compared to BLF (p<0.001, FIG. 3) with no significant increase over the negative BSA control (p>0.1; FIG. 3).

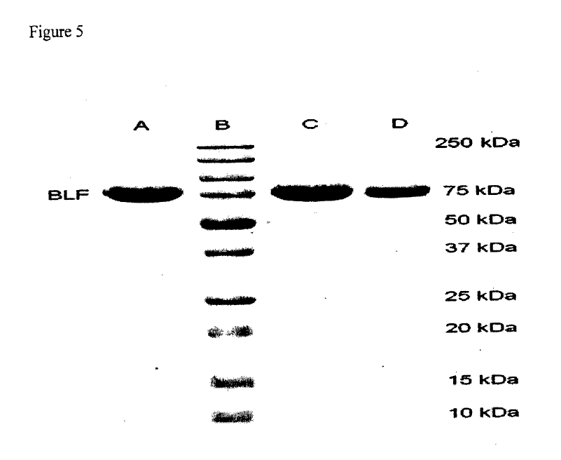

Comparison of the total protein content of the unbound and eluted fractions from the serine protease affinity column showed approximately 5% of native BLF bound to the benzamidine substrate. All fractions were the same apparent molecular weight as BLF by SDS-PAGE with no visible contaminating bands in the eluted fraction (FIG. 5).

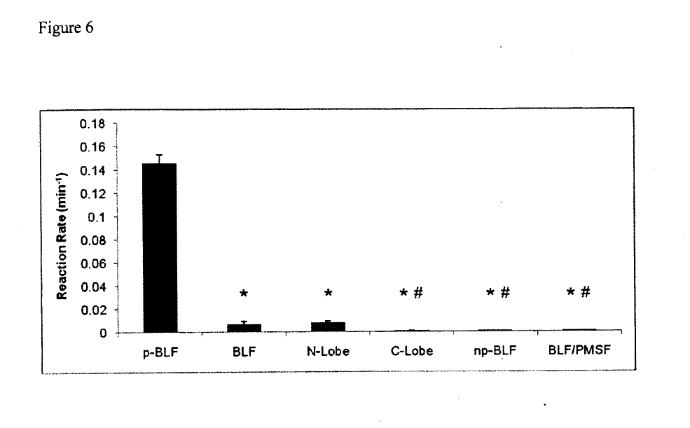

The proteolytic activity of BLF eluted from the benzamidine was found to have a Kₘ of 34±4 μM and a kₑ. 0.3±0.08 min⁻¹ for the serine protease substrate Z-Phe-Arg-AMC in pH 7.0 at 25° C. This fraction of BLF, proteolytic (p-BLF), had substantially greater proteolytic activity than native BLF or the unbound, non-proteolytic (np-BLF), BLF (p<0.005, FIG. 6). Hydrolysis of the serine protease substrate was found to be significantly greater by native BLF and the N-lobe (p<0.05, FIG. 6) compared to the C-lobe, np-BLF and PMSF inhibited BLF.

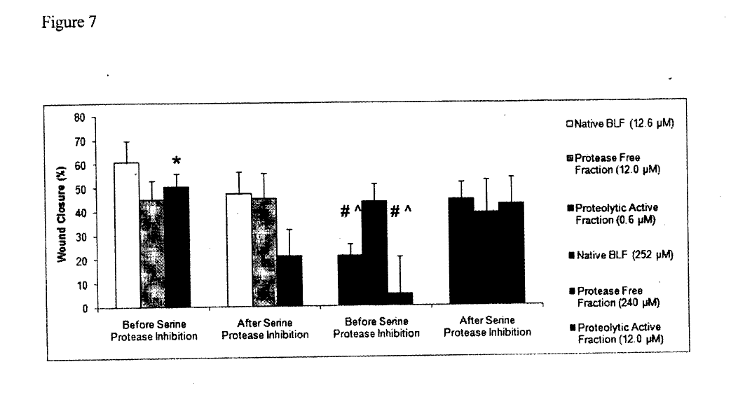

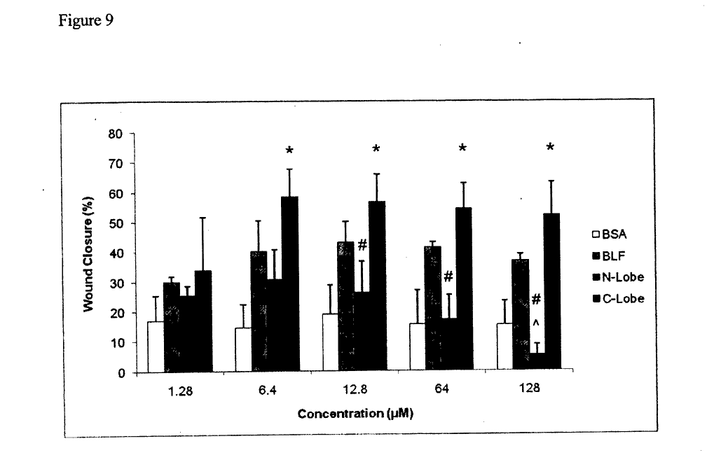

To determine the relative contributions of the p-BLF and np-BLF fractions to the promotion of wound healing by BLF they were initially tested at the concentrations, 0.6 μM and 12.0 μM respectively, estimated to be present in 12.6 μM native BLF. Wounds incubated with 0.6 μM p-BLF or 12.0 μM np-BLF produced a similar degree of wound closure (p>0.5, FIG. 7). This concentration of p-BLF was lower than that required for native BLF to promote wound closure (FIG. 9). Serine protease inhibition of the benzamidine column fractions at these concentrations only significantly reduced the promotion of wound healing for p-BLF (p<0.001, FIG. 7).

When the concentration of all fractions was increased 20 fold the healing response was markedly less for native BLF and p-BLF compared to their respective low concentration preparations (p<0.001, FIG. 7). Serine protease inhibition of these concentrations of native BLF and p-BLF restored the wound healing effect (p<0.005, FIG. 7) to the level of the np-BLF (p>0.5, FIG. 7).

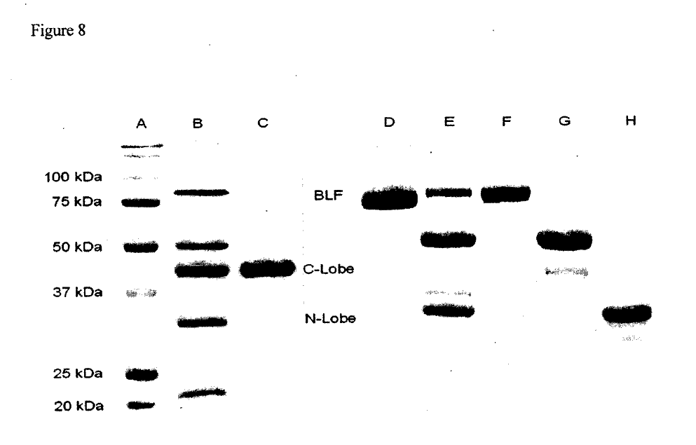

BLF subjected to limited tryptic digestion followed by ion-exchange and size exclusion chromatography was separated and purified into its N-lobe and C-lobe. Optical densitometry of bands visualised by SDS-PAGE of apparent molecular weight corresponding to BLF N-lobe and C-lobe accounted for over 90% of the protein present in their respective isolated fractions (FIG. 8).

The C-lobe promotes greater wound healing than equimolar levels of intact BLF and the N-lobe for concentrations 6.4 μM to 128 μM (p<0.05 and p<0.001, respectively; FIG. 9). At 6.4 μM the C-lobe promotes a 4 fold increase in wound closure over BSA compared to 3 fold for native BLF (FIG. 9). The N-lobe promotes less healing than intact BLF at concentrations of 12.8 μM to 128 μM (p<0.005, FIG. 9) with the only significant increase above BSA observed at 6.4 μM (p=0.014, FIG. 9). For N-lobe concentrations above this level wound closure becomes progressively less. At 128 μM the N-lobe promoted less wound closure than BSA (p<0.05, FIG. 9).

The following experiments in Guinea pigs show that the isolated C-Lobe promotes more rapid healing of corneal wounds in vivo than the vehicle, N-Lobe or whole BLF.

### Guinea Pig Debridement Wound: Method

Full thickness epithelial debridement wounds were created in the centre of the cornea by first demarking the area with a 3 mm diameter trephine and then gently scaping the epithelium away down to the basement membrane. These eyes were treated with 25 uL of either vehicle (PBS ph 7.4) or vehicle with 64 μM BLF or vehicle with 64 μM BLF N-Lobe or vehicle with 64 μM BLF C-Lobe. Each treatment group contained 9 guinea pigs with no significant difference in age, weight, or health. Dosing was immediately after debridement, then every three hours for the first 24 hours and then three times a day until completely healed. Wound closure was monitored by imaging the eye every 6 hours, in the presence of sodium fluorescein for contrast, until no staining was observed. Areas of wounds were calculated using lmageJ 1.44o (National Institutes of Health, USA) and then converted to an average wound diameter at each time point.

### Guinea Pig Alkali Wound: Method

Alkali burns of approximately 3 mm diameter were created in the centre of the cornea by application of a filter paper disc impregnated with 1 M sodium hydroxide for 20 seconds followed by extensive irrigation with saline. This removed the epithelium down to the basement membrane. These eyes were treated with 25 uL of either vehicle (PBS pH 7.4) or vehicle with 64 μM BLF or vehicle with 64 μM BLF N-Lobe or vehicle with 64 μM BLF C-Lobe. Each treatment group contained 9 guinea pigs with no significant difference in age, weight, or health. Dosing was immediately after irrigation, then every hour for the first 8 hours and then three times a day until completely healed. Wound closure was monitored by imaging the eye every 12 hours, in the presence of sodium fluorescein for contrast, until no staining was observed. Areas of wounds were calculated using ImageJ 1.44o (National Institutes of Health, USA) and then converted to an average wound diameter at each time point.

### Guinea Pig Models: Statistical Analysis

Results were analysed to determine differences between treatments within each time point using one-way analysis of variance followed by post hoc multiple comparisons with Bonferroni correction. Further analysis of the number of wounds completely closed at particular time points was by Fisher's exact test with comparison to the vehicle control and correction for multiple comparisons.

These in vitro experiments show the effect the isolated C-lobe has on wound healing related cellular activity.

### Cell Proliferation Assay: Method

Immortalised human comeolimbal epithelial (HCLE) cells were seeded at 40% confluence in 96 well tissue culture plates and allowed to attach overnight at 37° C. in, a 5% CO2 atmosphere. The next day the medium was replaced and supplemented with either bovine serum albumin (BSA) or BLF or BLF N-Lobe or BLF C-Lobe at concentrations of 1.28 μM, 6.4 μM, 12.8 μM, 64 μM and 128 μM, each with 8 replicates, and incubated for 24 hours. Cell proliferation was then measured by CyQuant Cell Proliferation Assay, Kit (Invitrogen, USA) according to the manufacturer's instructions. Briefly, the wells were emptied of medium and lysed by storage at −80° C. overnight. The next day the plates were thawed and 200 μL of CyQuant GR dye in cell lysis buffer was added to each well. Sample fluorescence, reflecting DNA levels, was then measured at an excitation wavelength of 480 nm and an emission wavelength of 520 nm.

Results were expressed as averages for each treatment at a concentration and compared to equimolar BSA by ANOVA with Bonferroni correction.

Cell Migration Assay: Method

Immortalised human comeolimbal epithelial (HCLE) cells were seeded at 100% confluence in 96 well Oris Cell Migration. Assay (Platypus Technologies, USA) tissue culture plates coated with fibronectin and allowed to attach overnight at 37° C. in a 5% CO2 atmosphere. In the morning the plugs were removed allowing the cells to migrate into the central 2 mm diameter area of the well. The medium was replaced and supplemented with 1 mM hydroxyurea to inhibit proliferation and either bovine serum albumin (BSA) or BLF or BLF N-Lobe or BLF C-Lobe at concentrations of 1.28 μM, 6.4 μM, 12.8 μM, 64 μM and 128 μM, each with 8 replicates, and incubated for 16 hours. Migration of the cells was monitored by fluorescent confocal microscopy using CellTracker Green CMFDA (Molecular Probes, USA) to stain the cytoplasm. Images were analysed using ImageJ 1.44o (National Institutes of Health, USA) to calculate the area of the wound remaining. The results were expressed as average area±standard deviation and compared to equimolar BSA by ANOVA with Bonferroni correction.

### Results

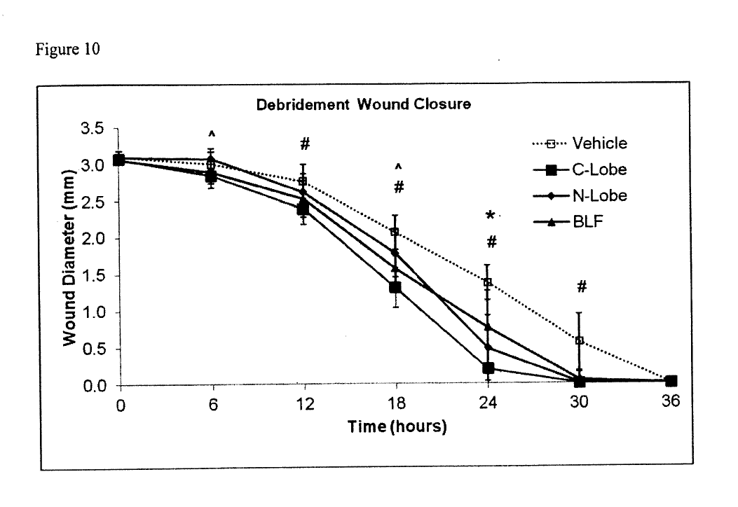

FIG. 10 shows the time course of wound closure in the guinea pig debridement model in which the isolated C-Lobe promoted more rapid healing than the vehicle, N-Lobe or whole BLF (Table 1). The C-lobe treated wounds are significantly smaller than those treated with vehicle only (p<0.005) by 12 hours and remain smaller until closure.

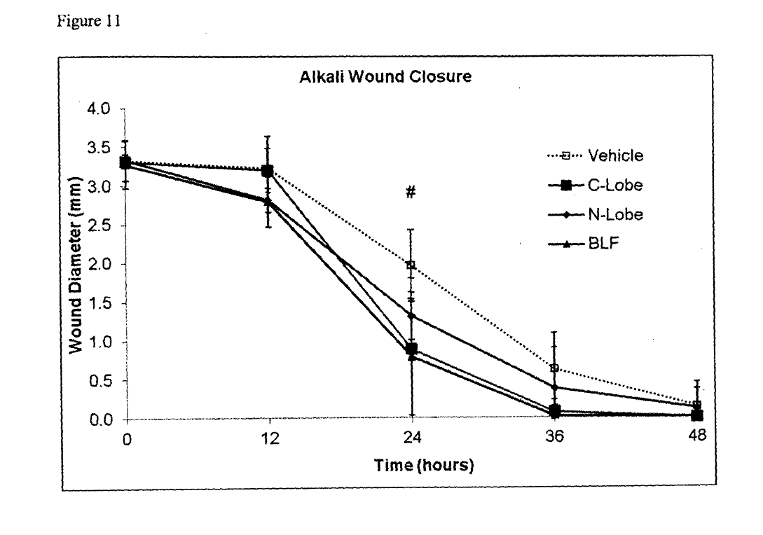

FIG. 11 shows the time course of wound closure in the guinea pig alkali burn model in which the isolated C-Lobe and whole BLF promoted more rapid healing than the vehicle or N-Lobe (Table I). Those wounds treated with C-lobe are significantly smaller than vehicle treated wounds at 24 hours (p=0.013).

TABLE 1
Wounds completely closed at 24 and 36 hours
after injury for debridement and alkali wounds
respectively. n = 9 for all groups.

| Debridement Wounds | | | | Alkali Wounds | | | |
|---|---|---|---|---|---|---|---|
| Closed at 24 hours | | | | Closed at 36 hours | | | |
| Vehicle | BLF | N-Lobe | C-Lobe | Vehicle | BLF | N-Lobe | C-Lobe |
| 0% | 22% | 33% | 67% | 0% | 89% | 44% | 78% |
| p-value | 1.0 | 0.6 | 0.03 | p-value | 0.001 | 0.2 | 0.007 |

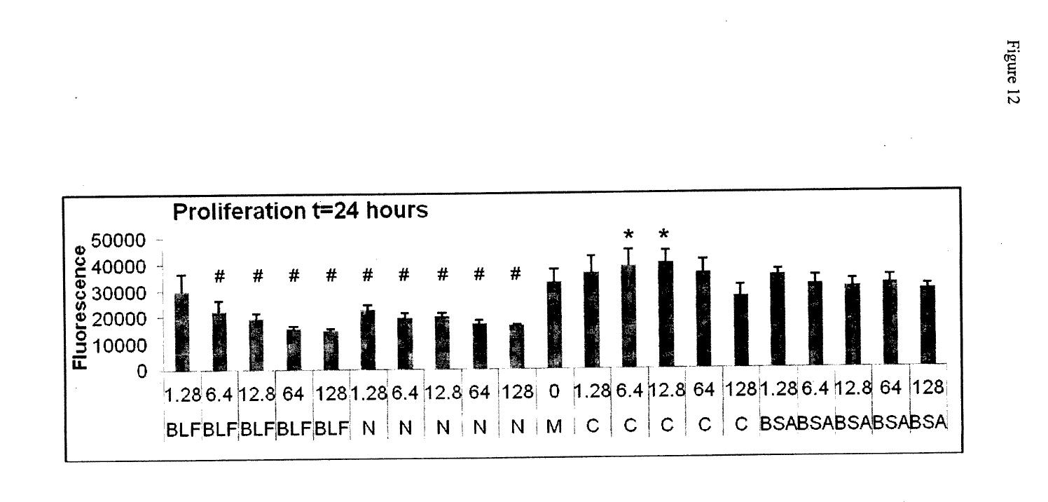

FIG. 12 shows that in vitro the C-Lobe at concentrations of 6.4 μM and 12.8 μM increases Human Corneolimbal Epithelial cell proliferation rates by 24 hours (p<0.001) while whole BLF and the N-Lobe in isolation reduce proliferation (p<0.05) at all concentrations with the exception of BLF at 1.28 μM which has no effect. All other C-Lobe concentration have no significant impact on proliferation relative to equimolar BSA.

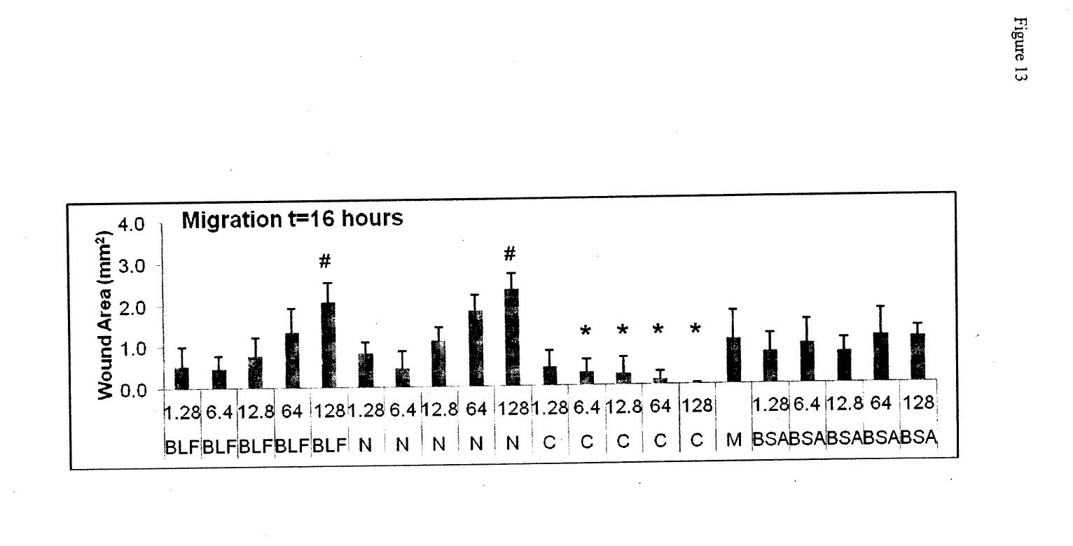

FIG. 13 shows that in vitro the C-Lobe increases the rate of migration of Human Corneolimbal Epithelial cells at 16 hours for concentrations at and above 6.4 μM while whole BLF and the N-Lobe show a concentration dependent slowing of cell migration that becomes significant at a concentration of 128 μM (p<0.001).

Thus the in vitro system indicates the C-lobe has a different effect on Human Corneolimbal Epithelial cells in terms of proliferation, migration and wound healing. In the guinea pig model the C-Lobe out performs whole BLF and the isolated N-Lobe in the debridement model while being as effective as whole BLF in the alkali burn model.

It will be understood that the invention disclosed and defined in this specification extends to all alternative combinations of two or more of the individual features mentioned or evident from the text or drawings. All of these different combinations constitute various alternative aspects of the invention.

1. A pharmaceutical composition comprising an effective amount of a polypeptide or peptidomimetic consisting essentially of the C-lobe of lactoferrin, or functionally active fragments or variants thereof.

2. A pharmaceutical composition according to claim 1, wherein the lactoferrin is bovine lactoferrin.

3. A pharmaceutical composition according to claim 1, wherein the polypeptide or peptidomimetic consists essentially of the amino acid sequence shown in SEQ ID NO: 1.

4. A pharmaceutical composition according to claim 1, wherein the polypeptide or peptidomimetic consists of the C-lobe of lactoferrin.

5. A pharmaceutical composition according to claim 1, wherein the functionally active fragment is a polypeptide or peptidomimetic having an amino acid sequence of greater than 30 amino acids in length and greater than 65% identity with a contiguous sequence of SEQ ID NO: 1.

6. A pharmaceutical composition according to claim 1, wherein the C-lobe is obtained by proteolysis of whole lactoferrin.

7. A pharmaceutical composition according to claim 1, which is in a form suitable for administration to the eye.

8. A pharmaceutical composition according to claim 1, which is an aqueous solution.

9. A pharmaceutical composition according to claim 1, wherein the composition is in the form of eye drops.

10. A method of treating a corneal wound comprising administering to a subject in need thereof a pharmaceutical composition according to claim 1.

11. A method according to claim 10, wherein the subject is a human patient.

12. A method according to claim 10, wherein the corneal wound is an epithelial corneal wound.

13. A method according to claim 12, wherein the epithelial corneal wound is an alkali-induced wound.

14. A method according to claim 10, wherein the subject has been identified as having a corneal wound.


SEQUENCE LISTING (1 sequence)

SEQ ID NO: 1 - protein, 348 aa, Bovine
YTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR